(19) United States
(12) Patent Application Publication
(10) Pub. No.: US 2015/0065821 A1
(43) Pub. Date: Mar. 5, 2015

(54) Nanoparticle Phoresis

(71) Applicant: Google Inc., Mountain View, CA (US)

(72) Inventor: Andrew Jason Conrad, Mountain View, CA (US)

(73) Assignee: Google Inc., Mountain View, CA (US)

(21) Appl. No.: 14/018,591

(22) Filed: Sep. 5, 2013

(51) Int. Cl. A61N 2/00

(52) U.S. Cl. CPC A61N 2/004
USPC 600/309; 600/12

(57) ABSTRACT

Functionalized particles in the blood are able to selectively bind to targets in the blood that have adverse health effects. The binding of the particles to the targets allows the targets to be selectively modified or destroyed by energy from outside the body such that the adverse health effects are reduced or eliminated. The energy is generated by a wearable device which is able to direct the energy into the subsurface vasculature of the wearer of the wearable device. Further, one or more of the functionalized particles may be magnetic, allowing a magnetic field generated by the wearable device and directed into the subsurface vasculature to concentrate the bound targets in a lumen of the subsurface vasculature proximate to the wearable device.

### BACKGROUND

Unless otherwise indicated herein, the materials described in this section are not prior art to the claims in this application and are not admitted to be prior art by inclusion in this section.

A number of scientific methods have been developed in the medical field to examine physiological conditions of a person, for example, by detecting and/or measuring one or more analytes in a person's blood. The one or more analytes could be any analytes that, when present in or absent from the blood, or present at a particular concentration or range of concentrations, may be indicative of a medical condition or health of the person. The one or more analytes could include enzymes, hormones, proteins, cells or other substances. In addition, a number of methods have been developed for preventing, treating or curing diseases or medical conditions by targeting certain blood analytes thought to be the cause or a contributing factor of a disease or condition. The methods attempt to deactivate, destroy or remove from the body the target blood analytes. In a typical scenario, a patient may be given a drug or subjected to an external energy source (laser, ultrasound, RF, X-rays, gamma rays, neutron sources), etc., that modifies or destroys the offensive analyte and/or tags it for removal from the body. However, these forms of treatment are often systemic and not specific to the target blood analyte, which may cause the death, removal, or modification of desirable analytes, such as healthy cells or proteins necessary for normal biological functions. Moreover, due to their lack of specificity or targeting, these known treatments may not destroy or remove all of the target analytes from the body, reducing the overall effectiveness of the treatment. Many of these known treatments also require a patient to travel to a hospital or medical setting and endure lengthy treatments.

### SUMMARY

Some embodiments of the present disclosure provide a wearable device, including: a mount configured to mount the wearable device to an external body surface proximate to a portion of subsurface vasculature; a magnet configured to direct a magnetic field into the portion of subsurface vasculature, in which the magnetic field is sufficient to cause functionalized magnetic particles to collect in a lumen of the portion of the subsurface vasculature, and the functionalized magnetic particles are configured to complex with a target that has an ability to cause an adverse health effect; and a signal source configured to transmit a signal into the portion of subsurface vasculature sufficient to cause a physical or chemical change in the target complexed with the functionalized magnetic particles, in which the physical or chemical change reduces or eliminates the target's ability to cause the adverse health effect.

Some embodiments of the present disclosure present a method, including: introducing functionalized magnetic particles into a lumen of subsurface vasculature, in which the functionalized magnetic particles are configured to complex with a target in blood circulating in the subsurface vasculature, the target having an ability to cause an adverse health effect; directing, from a magnet in the wearable device, a magnetic field into the subsurface vasculature proximate to the wearable device, in which the magnetic field is sufficient to cause the functionalized magnetic particles complexed with the target to collect in a lumen of the subsurface vasculature proximate to the wearable device; and directing, from a signal source in the wearable device, a signal into the portion of subsurface vasculature sufficient to cause a physical or chemical change in the target complexed with the functionalized magnetic particles, in which the physical or chemical change reduces or eliminates the target's ability to cause the adverse health effect.

Some embodiments of the present disclosure present a method, including: introducing a first type of functionalized particles into a lumen of subsurface vasculature, in which the functionalized particles of the first type are magnetic; introducing a second type of functionalized particles into a lumen of subsurface vasculature, in which the functionalized particles of the second type are configured to bind to a target in blood circulating in the subsurface vasculature, the target having an ability to cause an adverse health effect, and the functionalized particles of the first type are configured to bind to functionalized particles of the second type that are bound to the target; (iv) directing, from a magnet in the wearable device, a magnetic field into the subsurface vasculature proximate to the wearable device, in which the magnetic field is sufficient to cause functionalized particles of the first type bound to functionalized particles of the second type to collect in a lumen of the subsurface vasculature proximate to the wearable device; and (v) directing, from a signal source in the wearable device, a signal into the portion of subsurface vasculature sufficient to cause a physical or chemical change in the target bound to functionalized particles of the second type, in which the physical or chemical change reduces or eliminates the target's ability to cause the adverse health effect.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 is a perspective view of an example wearable device.

FIG. 2A is a perspective top view of an example wrist-mounted device, when mounted on a wearer's wrist.

FIG. 2B is a perspective bottom view of an example wrist-mounted device shown in FIG. 2A, when mounted on a wearer's wrist.

FIG. 3A is a perspective bottom view of an example wrist-mounted device, when mounted on a wearer's wrist.

FIG. 3B is a perspective top view of an example wrist-mounted device shown in FIG. 3A, when mounted on a wearer's wrist.

FIG. 3C is a perspective view of an example wrist-mounted device shown in FIGS. 3A and 3B.

FIG. 4A is a perspective view of an example wrist-mounted device.

FIG. 4B is a perspective bottom view of an example wrist-mounted device shown in FIG. 4A.

FIG. 5 is a perspective view of an example wrist-mounted device.

FIG. 6 is a perspective view of an example wrist-mounted device.

FIG. 7 is a block diagram of an example system that includes a plurality of wrist mounted devices in communication with a server.

FIG. 8 is a functional block diagram of an example wearable device.

FIG. 9 is a functional block diagram of an example wearable device.

FIG. 10 is a functional block diagram of an example wearable device.

FIG. 11A is side partial cross-sectional view of an example wrist-mounted device, while on a human wrist.

FIG. 11B is side partial cross-sectional view of an example wrist-mounted device, while on a human wrist.

FIG. 12A is side partial cross-sectional view of an example wrist-mounted device, while on a human wrist.

FIG. 12B is side partial cross-sectional view of an example wrist-mounted device, while on a human wrist.

FIG. 13A is side partial cross-sectional view of an example wrist-mounted device, while on a human wrist.

FIG. 13B is side partial cross-sectional view of an example wrist-mounted device, while on a human wrist.

FIG. 14A is side partial cross-sectional view of an example wrist-mounted device, while on a human wrist.

FIG. 14B is side partial cross-sectional view of an example wrist-mounted device, while on a human wrist.

FIG. 15 is a flowchart of an example method for modifying a target in a subsurface vasculature.

FIG. 16 is a flowchart of an example method for modifying a target in a subsurface vasculature.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

### I. OVERVIEW

A wearable device can automatically modify or destroy one or more targets in the blood that have an adverse health effect by transmitting energy into subsurface vasculature proximate to the wearable device. The targets could be any substances or objects that, when present in the blood, or present at a particular concentration or range of concentrations, may affect a medical condition or the health of the person wearing the device. For example, the targets could include enzymes, hormones, proteins, cells or other molecules. Modifying or destroying the targets could include causing any physical or chemical change in the targets such that the ability of the targets to cause the adverse health effect is reduced or eliminated.

The wearable device can include a mount that is configured to mount the device to a specific surface of the person's body, more particularly, to a body location where subsurface vasculature is readily affected. For example, the wearable device can include a wristband for mounting the wearable device on the wrist. In this position, the wearable device may be only about 2-4 millimeters away from the midpoint of an artery, capillary or vein in the wrist.

In an example embodiment, the wearable device changes the target by transmitting energy to functionalized particles which are bound to the target. The functionalized particles could be, for example, microparticles or nanoparticles that have been introduced into a lumen of the subsurface vasculature. The terms “binding” and “bound” is to be understood in the broadest sense to include any functional interaction between the target and the functionalized particles.

The functionalized particles can have a diameter that is less than about 20 micrometers. In some embodiments, the particles have a diameter on the order of about 10 nm to 1 μm. In further embodiments, small particles on the order of 10-100 nm in diameter may be assembled to form a larger “clusters” or “assemblies on the order of 1-10 micrometers. Those of skill in the art will understand a “particle” in its broadest sense and that it may take the form of any fabricated material, a molecule, tryptophan, a virus, a phage, etc. Further, a particle may be of any shape, for example, spheres, rods, non-symmetrical shapes, etc.

In some examples, the particles may be magnetic and can be formed from a paramagnetic, super-paramagnetic or ferromagnetic material or any other material that responds to a magnetic field. Alternatively, the particles may be made of non-magnetic materials, such as polystyrene, coupled with a magnetic material or moiety.

The transmitted energy can be any of a variety of types, including a radio frequency pulse, a time-varying magnetic field, an acoustic pulse, an infrared or visible light signal, or other types of directed energy which can be generated by a wearable device familiar to one of skill in the art. The energy is able to be specifically applied to the target due to the binding of the target to one or more functionalized particles. In one example, one of the functionalized particles is a functionalized magnetic particle; the wearable device could transmit a radio frequency (RF) pulse which could cause the magnetic particle to vibrate, and this vibration could cause localized heating of a target bound to the particle. This localized heating could cause the target to be denatured, lysed, or otherwise changed such that the target's adverse health effect is reduced.

The change in the target could be any alteration of the physical or chemical properties of the target such that the adverse health effect is reduced or eliminated. If the target is a protein or nucleic acid, the change could include altering the secondary, tertiary or quaternary structure of the target or even directly changing or breaking apart the primary structure of the target. If the target is a cell, changing could include damaging the cell wall to induce death of the cell or other changes to alter the function of the cell. The target may still exhibit some of the adverse health effect after modification or destruction, but the degree of the adverse health effect will be less than before the modification or destruction. In some cases, the modified or destroyed target may cause side effects or adverse health effects different from the original adverse health effect.

The particles, or a group of several particles in a complex, may be functionalized with a receptor that has a specific affinity to bind to or interact with a clinically relevant target. The receptor may be inherent to the particle itself. For example, the particle itself may be a virus or a phage with an inherent affinity for certain targets. Additionally or alternatively, the particles can be functionalized by covalently attaching a receptor that specifically binds or otherwise recognizes a particular clinically-relevant target. The functionalized receptor can be an antibody, peptide, nucleic acid, phage, bacteria, virus, or any other molecule with a defined affinity for a target analyte. Other compounds or molecules, such as fluorophores or autofluorescent or luminescent markers, which may assist in changing the target or interrogating the particles in vivo, may also be attached to the particles.

The functionalized particles can be introduced into the person's blood stream by injection, ingestion, inhalation, transdermal application, or in some other manner. Where magnetic particles are used, the wearable device may include a magnet that can direct into the portion of subsurface vasculature a magnetic field that is sufficient to cause the functionalized magnetic particles to collect in a lumen of the portion of subsurface vasculature. However, modification of the targets may be effected without localized “collection” of the functionalized particles. The wearable device may be configured to activate the magnet periodically, such as at certain times of every day (e.g., every hour). The collection of the particles proximate to the wearable device could be done to reduce the amount of energy transmitted from the wearable device necessary to modify or destroy the target.

The wearable device may further include one or more data collection systems for interrogating, in a non-invasive manner, the functionalized particles present in a lumen of the subsurface vasculature proximate to the wearable device. In one example, the wearable device includes a signal source for transmitting an interrogating signal that can penetrate into the portion of subsurface vasculature and a detector for detecting a response signal that is transmitted from the portion of subsurface vasculature in response to the interrogating signal. The interrogating signal can be any kind of signal that results in a response signal that can be used to detect binding of the clinically-relevant target to the functionalized particles. In one example, the interrogating signal is a radio frequency (RF) signal and the response signal is another RF signal or a magnetic resonance signal, such as nuclear magnetic resonance (NMR). In another example, where the functionalized particles include a fluorophore, the interrogating signal is an optical signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector. In some cases, the interrogating signal is the signal which is used to modify or destroy the target.

Further, in some cases, an interrogating signal may not be necessary to produce a response signal. For example, where the functionalized particles include an autofluorescent or luminescent marker, an interrogating signal may not be necessary. In some examples, the functionalized particles may include a chemo-luminescent marker configured to produce a response signal in the form of fluorescence radiation produced in response to a chemical reaction initiated, at least in part, by the binding of the target to the particle.

The wearable device can also include one or more data collection systems that do not make use of functionalized particles. For example, the wearable device can include sensors for measuring blood pressure, pulse rate, skin temperature, or other parameters. If in the form of a wristband, the wearable device may also include a watch face for displaying the time and/or date.

In addition, the wearable device may be configured to analyze the data that it collects. For example, the wearable device may include a computing device that is configured to detect the presence or absence of the clinically-relevant target based on a detected response signal and, in some examples, to further determine a concentration of the clinically-relevant target based on the detected response signal and determine whether a medical condition is indicated based on at least the presence, absence and/or concentration of the clinically-relevant target. The wearable device may also include a user interface that can display the results of the data analysis, such as whether the clinically-relevant target is present and in what concentration. In this way, the person wearing the device can be made aware of medical conditions in real time. The wearable device may also be configured to produce an auditory or tactile (vibration) response to alert the person wearing the device of a medical condition. The device could also use the information about the target to determine when and how to activate the signal source to modify or destroy the target. For example, when the detected level of the target exceeds some threshold, a signal could be transmitted to modify or destroy the target until the concentration of the target is reduced to a second threshold level less than the first threshold level.

The wearable device may further include a communication interface for transmitting the results of the data analysis and the modification of the target to medical personnel and/or receiving instructions or recommendations based on a medical personnel or remote computing device's interpretation of those results. In some examples, the communication interface is a wireless communication interface. The communication interface may also include a universal serial bus (USB) interface, a secure digital (SD) card interface, a wired interface, or any other appropriate interface for communicating data from the device to a server. The term “server” may include any system or device that responds to requests across a computer network to provide, or helps to provide, a network service, and may include servers run on dedicated computers, mobile devices, and those operated in a cloud computing network.

The wearable device may modify the target in each of a plurality of modification periods. The length of the modification period may be set on the device itself or may be set remotely, for example, by instruction from a remote server. The device may be configured with many modification periods each day—for example, continuous, every second, every minute, every hour, every 6 hours, etc.—according to an expected level of the target in the blood, rate of destruction of the target by the wearable device, and/or rate of creation of the target in the wearer's body.

Further, the wearable device may be configured to accept inputs from the wearer regarding his or her health state. The inputs may be subjective indicia regarding how the person is feeling or any symptoms he or she is experiencing at that time, such as, “feeling cold,” “feeling tired,” “stressed,” “feeling rested and energetic,” “pollen allergy symptoms today,” etc. Such inputs from the user may be used to complement any other physiological parameter data that the wearable device may collect and establish effective signal levels for and timing of modification of the target.

It should be understood that the above embodiments, and other embodiments described herein, are provided for explanatory purposes, and are not intended to be limiting. Further, the term “medical condition” as used herein should be understood broadly to include any disease, illness, disorder, injury, condition or impairment—e.g., physiologic, psychological, cardiac, vascular, orthopedic, visual, speech, or hearing—or any situation requiring medical attention.

### II. EXAMPLE WEARABLE DEVICES

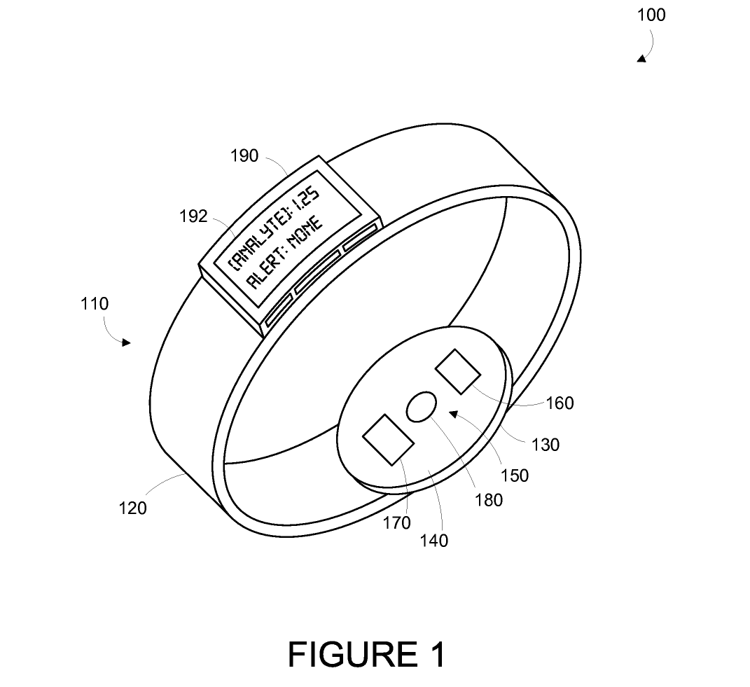

A wearable device 100 can automatically modify a plurality of targets and measure a plurality of physiological parameters of a person wearing the device. The term “wearable device,” as used in this disclosure, refers to any device that is capable of being worn at, on or in proximity to a body surface, such as a wrist, ankle, waist, chest, or other body part. In order to modify targets and take in vivo measurements in a non-invasive manner from outside of the body, the wearable device may be positioned on a portion of the body where subsurface vasculature is easily affectable and observable, depending on the type of modification and detection systems used. The device may be placed in close proximity to the skin or tissue, but need not be touching or in intimate contact therewith. A mount 110, such as a belt, wristband, ankle band, etc. can be provided to mount the device at, on or in proximity to the body surface. The mount 110 may prevent the wearable device 100 from moving relative to the body to ensure effective modification of the target. In one example, shown in FIG. 1, the mount 110, may take the form of a strap or band 120 that can be worn around a part of the body. Further, the mount 110 may include an adhesive material for adhering the wearable device 100 to the body of a wearer.

A modification platform 130 is disposed on the mount 110 such that it can be positioned on the body where subsurface vasculature is easily affected. An inner face 140 of the modification platform is intended to be mounted facing to the body surface. The modification platform 130 may house a modification system 150, which may include at least one transmitter 160 for modifying at least one target. For example, the target may be bound to a functionalized particle, and the activity of the transmitter 160 may excite the functionalized particle such that a physical or chemical change is caused in the target. This change reduces the target's ability to cause an adverse health effect. In a non-exhaustive list, the transmitter 160 may include any of an optical (e.g., LED, laser), acoustic (e.g., piezoelectric, piezoceramic), thermal, magnetic, or electromagnetic (e.g., RF, magnetic resonance) transmitter. The change in the target may be due to coupling of energy from the transmitter through the functionalized particle or may be due to energy directly applied to the target. In one example, the functionalized particles could be magnetic and could have a resonance frequency. Transmitting from the transmitter 160 an RF pulse or time-varying magnetic field at the resonance frequency of the particles could result in localized heating of the target, causing the modification or destruction of the target. In another example, the transmitter 160 transmits a light pulse, which causes a localized heating of the target due to a photoacoustic effect. There may be more than one type of functionalized particle bound to the target; for example, one functionalized particle may bind to the target, causing it to change a shape such that a second functionalized particle is able to bind to the changed shape of the target. The components of the modification system 150 may be miniaturized so that the wearable device may be worn on the body without significantly interfering with the wearer's usual activities.

In some examples, the modification system 150 further includes at least one detector 170 for detecting at least one physiological parameter, which could include any parameters that may relate to the health of the person wearing the wearable device. For example, the detector 170 could be configured to measure blood pressure, pulse rate, respiration rate, skin temperature, etc. At least one of the detectors 170 could be configured to non-invasively measure one or more targets in blood circulating in subsurface vasculature proximate to the wearable device. In a non-exhaustive list, detector 170 may include any one of an optical (e.g., CMOS, CCD, photodiode), acoustic (e.g., piezoelectric, piezoceramic), electrochemical (voltage, impedance), thermal, mechanical (e.g., pressure, strain), magnetic, or electromagnetic (e.g., RF, magnetic resonance) sensor.

In some examples, the modification signal transmitter 160 is configured to transmit an interrogating signal that can penetrate into the portion of subsurface vasculature, for example, into a lumen of the subsurface vasculature. The interrogating signal can be any kind of signal, such as electromagnetic, magnetic, optic, acoustic, thermal, mechanical, that results in a response signal that can be used to measure a physiological parameter or, more particularly, that can detect the binding of the clinically-relevant target to the functionalized particles. In one example, the interrogating signal is an electromagnetic pulse (e.g., a radio frequency (RF) pulse) and the response signal is another RF signal or a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the particles by physical motion in a manner different from the background, making them easier to detect. In a further example, the interrogating signal is an electromagnetic radiation signal. In some examples, the functionalized particles include a fluorophore. The interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector. In some examples, this interrogation signal is the same as the modification signal used to change the target. In other examples, the interrogation signal is generated by an interrogation signal source different from the signal source which generates the target modification signal.

In some cases, the interrogation signal is not necessary to measure one or more of the physiological parameters. For example, the functionalized particles include an autofluorescent or luminescent marker, such as a fluorophore, that will automatically emit a response signal indicative of the binding of the clinically-relevant target to the functionalized particles, without the need for a modifying or interrogating signal or other external stimulus. In some examples, the functionalized particles may include a chemo-luminescent marker configured to produce a response signal in the form of fluorescence radiation produced in response to a chemical reaction initiated, at least in part, to the binding of the target analyte to the particle.

A collection magnet 180 may also be included in the modification system 150. In such embodiments, the functionalized particles may also be made of or be functionalized with magnetic materials, such as ferromagnetic, paramagnetic, super-paramagnetic, or any other material that responds to a magnetic field. The collection magnet 180 is configured to direct a magnetic field into the portion of subsurface vasculature that is sufficient to cause functionalized magnetic particles to collect in a lumen of that portion of subsurface vasculature. The magnet may be an electromagnet that may be turned on during a period in which a target is being measured and/or modified and turned off when the period is complete so as to allow the magnetic particles to disperse through the vasculature.

The wearable device 100 may also include a user interface 190 via which the wearer of the device may receive one or more recommendations or alerts generated from a remote server or other remote computing device, or from a processor within the device. The alerts could be any indication that can be noticed by the person wearing the wearable device. For example, the alert could include a visual component (e.g., textual or graphical information on a display), an auditory component (e.g., an alarm sound), and/or tactile component (e.g., a vibration). Further, the user interface 190 may include a display 192 where a visual indication of the alert or recommendation may be displayed. The display 192 may further be configured to provide an indication the battery status of the device or the status of the modification system or an indication of any measured physiological parameters, for instance, the concentrations of certain blood analytes being measured.

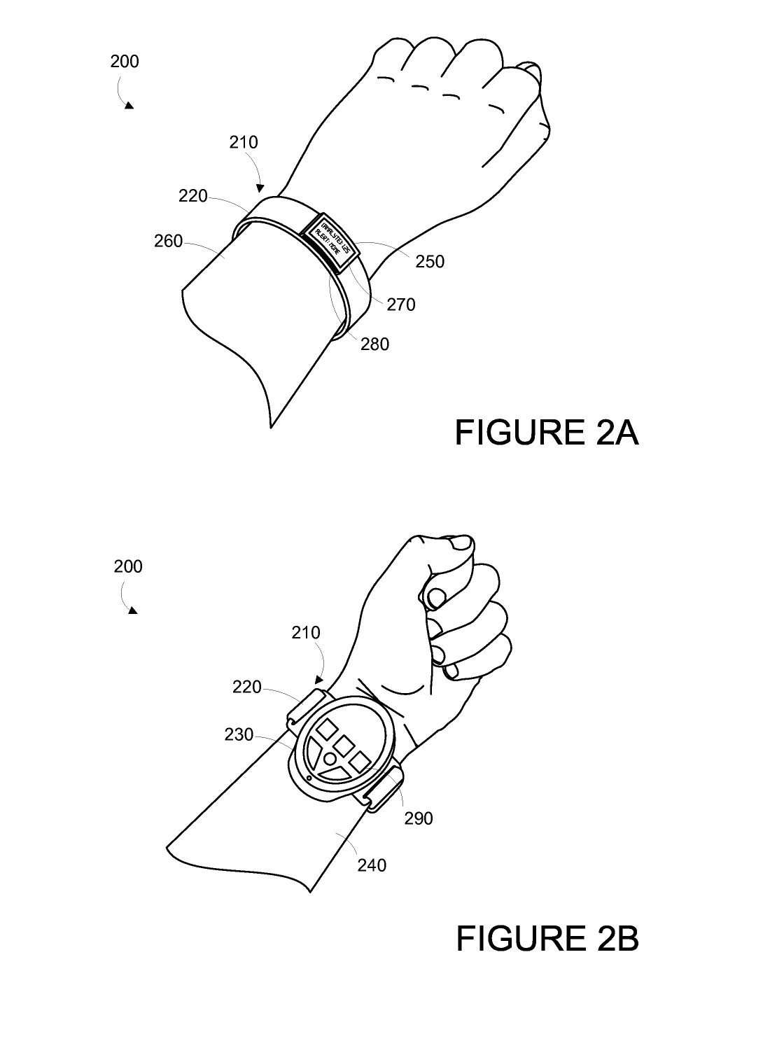

In one example, the wearable device is provided as a wrist-mounted device, as shown in FIGS. 2A, 2B, 3A-3C, 4A, 5B, 6 and 7. The wrist-mounted device may be mounted to the wrist of a living subject with a wristband or cuff, similar to a watch or bracelet. As shown in FIGS. 2A and 2B, the wrist mounted device 200 may include a mount 210 in the form of a wristband 220, a modification platform 230 positioned on the anterior side 240 of the wearer's wrist, and a user interface 250 positioned on the posterior side 260 of the wearer's wrist. The wearer of the device may receive, via the user interface 250, one or more recommendations or alerts generated either from a remote server or other remote computing device, or alerts from the modification platform. Such a configuration may be perceived as natural for the wearer of the device in that it is common for the posterior side 260 of the wrist to be observed, such as the act of checking a wrist-watch. Accordingly, the wearer may easily view a display 270 on the user interface. Further, the modification platform 230 may be located on the anterior side 240 of the wearer's wrist where the subsurface vasculature may be readily affectable. However, other configurations are contemplated.

The display 270 may be configured to display a visual indication of the alert or recommendation and/or an indication of the status of the wearable device and the modification of the target or an indication of measured physiological parameters, for instance, the concentrations of certain target blood analytes being modified. Further, the user interface 250 may include one or more buttons 280 for accepting inputs from the wearer. For example, the buttons 280 may be configured to change the text or other information visible on the display 270. As shown in FIG. 2B, measurement platform 230 may also include one or more buttons 290 for accepting inputs from the wearer. The buttons 290 may be configured to accept inputs for controlling aspects of the modification system, such as initiating a modification period, or inputs indicating the wearer's current health state (i.e., normal, migraine, shortness of breath, heart attack, fever, “flu-like” symptoms, food poisoning, etc.).

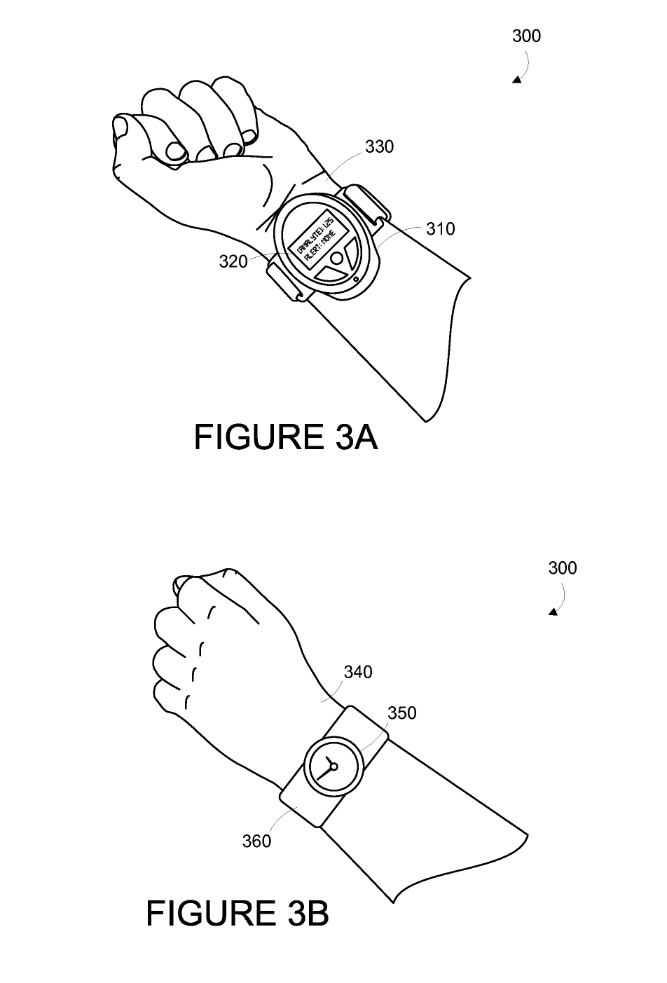

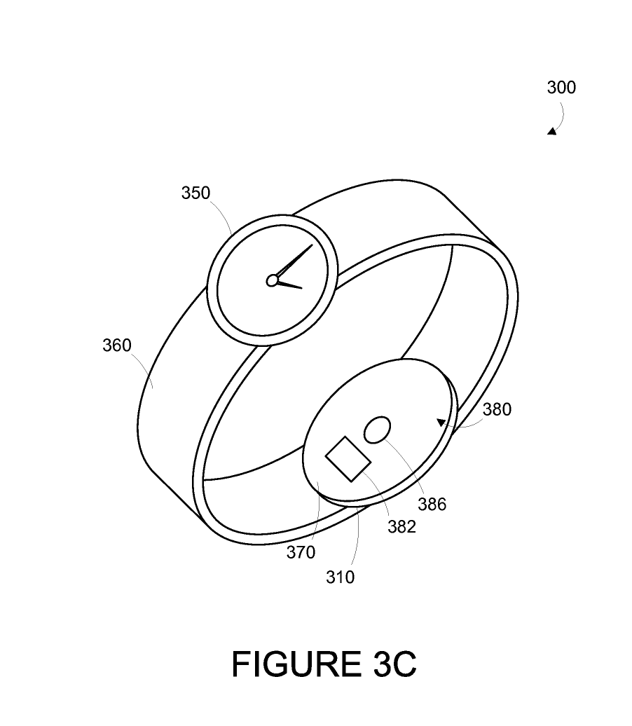

In another example wrist-mounted device 300, shown in FIGS. 3A-3C, the modification platform 310 and user interface 320 are both provided on the same side of the wearer's wrist, in particular, the anterior side 330 of the wrist. On the posterior side 340, a watch face 350 may be disposed on the strap 360. While an analog watch is depicted in FIG. 3B, one of ordinary skill in the art will recognize that any type of clock may be provided, such as a digital clock.

As can be seen in FIG. 3C, the inner face 370 of the modification platform 310 is intended to be worn proximate to the wearer's body. A modification system 380 housed on the measurement platform 310 may include a transmitter 382, and a collection magnet 386. As described above, the collection magnet 386 may not be provided in all embodiments of the wearable device.

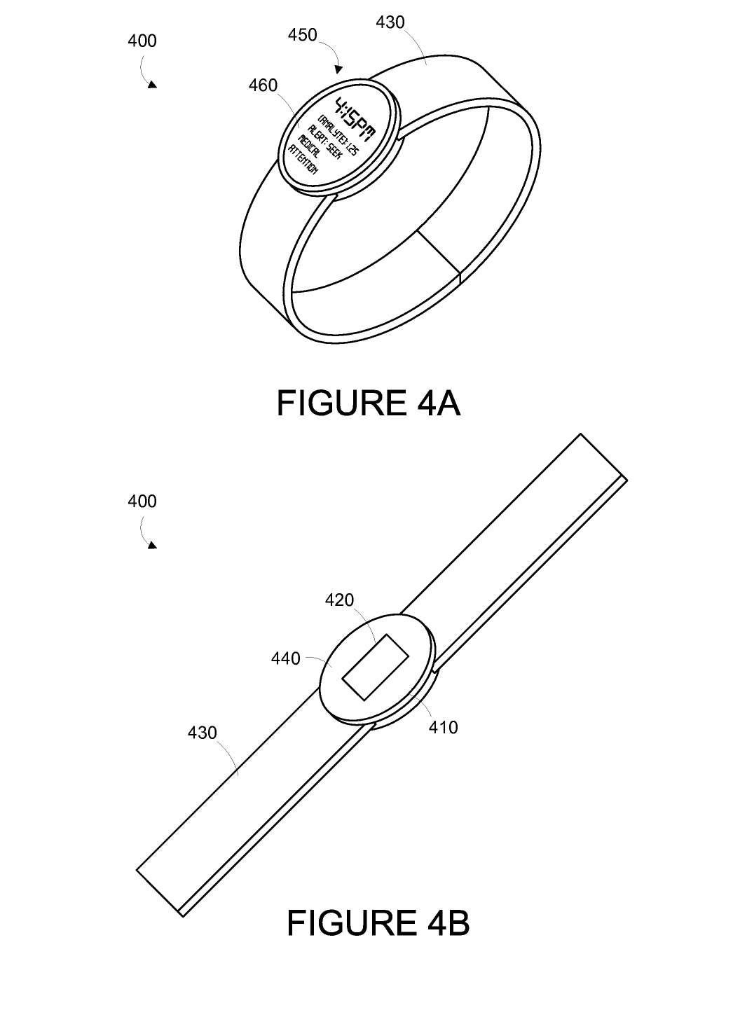

In a further example shown in FIGS. 4A and 4B, a wrist mounted device 400 includes a modification platform 410, which includes a modification system 420, disposed on a strap 430. Inner face 440 of modification platform 410 may be positioned proximate to a body surface so that modification system 420 may affect the subsurface vasculature of the wearer's wrist. A user interface 450 with a display 460 may be positioned facing outward from the modification platform 410. As described above in connection with other embodiments, user interface 450 may be configured to display data about the modification system 420, including the whether the modification system is active, and one or more alerts generated by a remote server or other remote computing device, or a processor located on the modification platform. The user interface 420 may also be configured to display the time of day, date, or other information that may be relevant to the wearer.

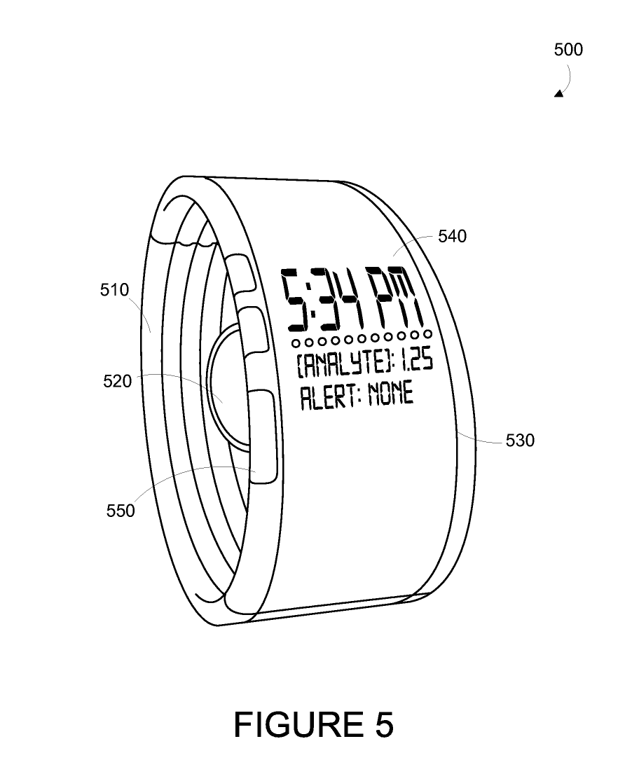

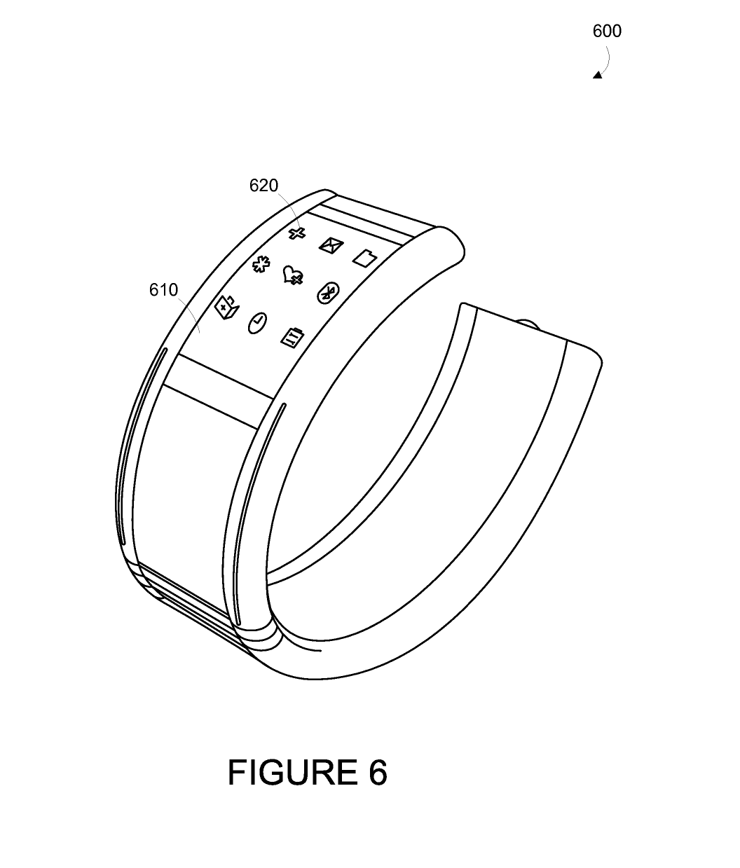

As shown in FIG. 5, in a further embodiment, wrist-mounted device 500 may be provided on a cuff 510. Similar to the previously discussed embodiments, device 500 includes a modification platform 520 and a user interface 530, which may include a display 540 and one or more buttons 550. The display 540 may further be a touch-screen display configured to accept one or more input by the wearer. For example, as shown in FIG. 6, display 610 may be a touch-screen configured to display one or more virtual buttons 620 for accepting one or more inputs for controlling certain functions or aspects of the device 600, or inputs of information by the user, such as current health state.

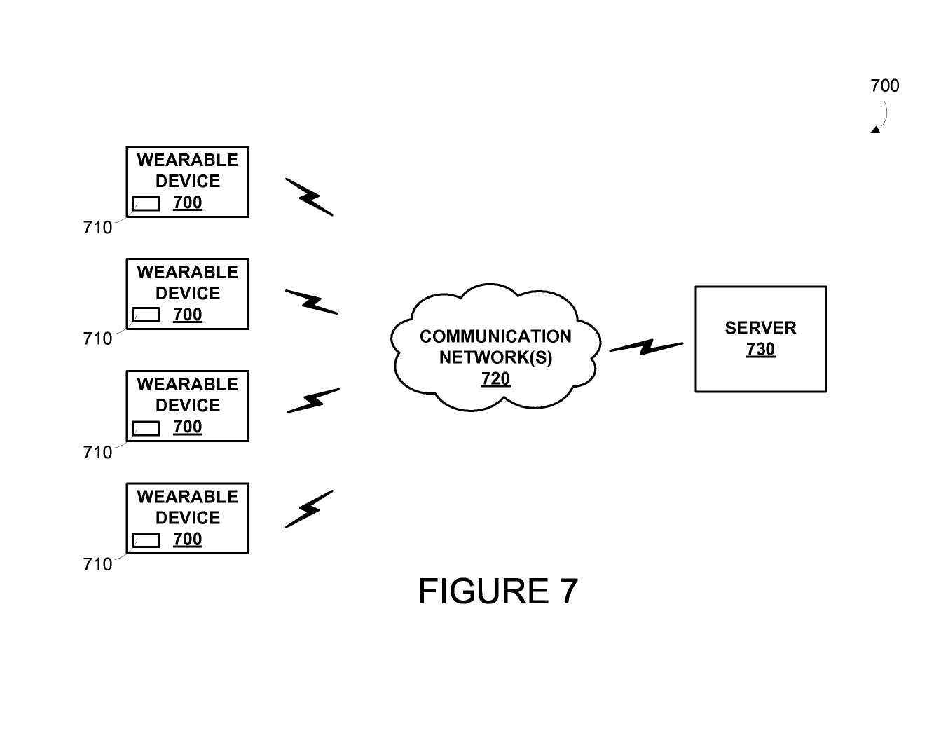

FIG. 7 is a simplified schematic of a system including one or more wearable devices 700. The one or more wearable devices 700 may be configured to transmit data via a communication interface 710 over one or more communication networks 720 to a remote server 730. In one embodiment, the communication interface 710 includes a wireless transceiver for sending and receiving communications to and from the server 730. In further embodiments, the communication interface 710 may include any means for the transfer of data, including both wired and wireless communications. For example, the communication interface may include a universal serial bus (USB) interface or a secure digital (SD) card interface. Communication networks 720 may include any of: a plain old telephone service (POTS) network, a cellular network, a fiber network and a data network. The server 730 may include any type of remote computing device or remote cloud computing network. Further, communication network 720 may include one or more intermediaries, including, for example wherein the wearable device 700 transmits data to a mobile phone or other personal computing device, which in turn transmits the data to the server 730.

In addition to receiving communications from the wearable device 700, such as data regarding health state as input by the user, the server may also be configured to gather and/or receive either from the wearable device 700 or from some other source, information regarding a wearer's overall medical history, environmental factors and geographical data. For example, a user account may be established on the server for every wearer that contains the wearer's medical history. Moreover, in some examples, the server 730 may be configured to regularly receive information from sources of environmental data, such as viral illness or food poisoning outbreak data from the Centers for Disease Control (CDC) and weather, pollution and allergen data from the National Weather Service. Further, the server may be configured to receive data regarding a wearer's health state from a hospital or physician. Such information may be used in the server's decision-making process, such as recognizing correlations and in generating clinical protocols or determining the timing and duration of target modification periods.

Additionally, the server may be configured to gather and/or receive the date, time of day and geographical location of each wearer of the device during each measurement period. If measuring physiological parameters of the user, such information may be used to detect and monitor spatial and temporal spreading of diseases. As such, the wearable device may be configured to determine and/or provide an indication of its own location. For example, a wearable device may include a GPS system so that it can include GPS location information (e.g., GPS coordinates) in a communication to the server. As another example, a wearable device may use a technique that involves triangulation (e.g., between base stations in a cellular network) to determine its location. Other location-determination techniques are also possible.

The server may also be configured to make determinations regarding the efficacy of a drug, functionalized magnetic particle, target modification signal, or other treatment based on information regarding the drugs or other treatments received by a wearer of the device and, at least in part, the physiological parameter data and the indicated health state of the user. From this information, the server may be configured to derive an indication of the effectiveness of the drug, functionalized magnetic particle, target modification signal or other treatment. For example, if the modification of the target is intended to reduce joint pain and the wearer of the device does not indicate that he or she is experiencing joint pain after transmitting a modification signal, the server may be configured to derive an indication that the level of transmitted modification signal is sufficient for that wearer.

Further, some embodiments of the system may include privacy controls which may be automatically implemented or controlled by the wearer of the device. For example, where a wearer's collected data are uploaded to a cloud computing network for analysis by a clinician, the data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined.

Additionally or alternatively, wearers of a device may be provided with an opportunity to control whether or how the device collects information about the wearer (e.g., information about a user's medical history, social actions or activities, profession, a user's preferences, or a user's current location), or to control how such information may be used. Thus, the wearer may have control over how information is collected about him or her and used by a clinician or physician or other user of the data. For example, a wearer may elect that data, such as health state and physiological parameters, collected from his or her device may only be used for generating an individual baseline and recommendations in response to collection and comparison of his or her own data and may not be used in generating a population baseline or for use in population correlation studies.

### III. EXAMPLE ELECTRONICS PLATFORM FOR A WEARABLE DEVICE

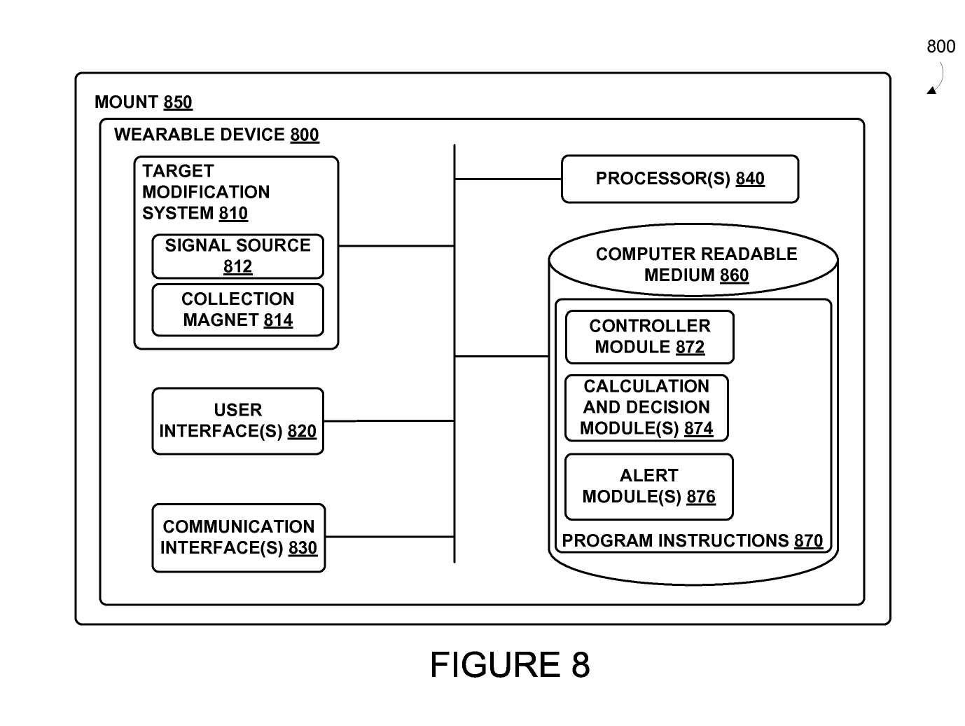

FIG. 8 is a simplified block diagram illustrating the components of a wearable device 800, according to an example embodiment. Wearable device 800 may take the form of or be similar to one of the wrist-mounted devices 200, 300, 400, 500, 600, shown in FIGS. 2A-B, 3A-3C, 4A-4C, 5 and 6. However, wearable device 800 may also take other forms, for example, an ankle, waist, or chest-mounted device.

In particular, FIG. 8 shows an example of a wearable device 800 having a target modification system 810, a user interface 820, communication interface 830 for transmitting data to a server, and processor(s) 840. The components of the wearable device 800 may be disposed on a mount 850 for mounting the device to an external body surface where a portion of subsurface vasculature is readily observable.

Processor 840 may be a general-purpose processor or a special purpose processor (e.g., digital signal processors, application specific integrated circuits, etc.). The one or more processors 840 can be configured to execute computer-readable program instructions 870 that are stored in a computer readable medium 860 and are executable to provide the functionality of a wearable device 800 described herein.

The computer readable medium 860 may include or take the form of one or more non-transitory, computer-readable storage media that can be read or accessed by at least one processor 840. The one or more computer-readable storage media can include volatile and/or non-volatile storage components, such as optical, magnetic, organic or other memory or disc storage, which can be integrated in whole or in part with at least one of the one or more processors 840. In some embodiments, the computer readable medium 860 can be implemented using a single physical device (e.g., one optical, magnetic, organic or other memory or disc storage unit), while in other embodiments, the computer readable medium 860 can be implemented using two or more physical devices.

Target modification system 810 includes a signal source 812 and, in some embodiments, a collection magnet 814. As described above, signal source 812 may include any element capable of causing a physical or chemical change in a target that has the ability to cause an adverse health effect. For example, the target may be bound to a functionalized particle, and the activity of the signal source 812 may excite the functionalized particle such that a physical or chemical change is caused in the target. This change reduces the target's ability to cause the adverse health effect. In a non-exhaustive list, the signal source 812 may include any of an optical (e.g., LED, laser), acoustic (e.g., piezoelectric, piezoceramic), thermal, magnetic, or electromagnetic (e.g., RF, magnetic resonance) transmitter. The change in the target may be due to coupling of energy from the transmitter through the functionalized particle or may be due to energy directly applied to the target. In one example, the functionalized particles could be magnetic and could have a resonance frequency. Transmitting from the transmitter 160 an RF pulse or time-varying magnetic field at the resonance frequency of the particles could result in localized heating of the target, causing the modification or destruction of the target. In another example, the transmitter 160 transmits a light pulse, which causes a localized heating of the target due to a photoacoustic effect. There may be more than one type of functionalized particle bound to the target; for example, one functionalized particle may bind to the target, causing it to change a shape such that a second functionalized particle is able to bind to the changed shape of the target. In this example, the collection magnet 814 may be used to locally collect functionalized magnetic particles present in an area of subsurface vasculature proximate to the collection magnet 814.

The program instructions 870 stored on the computer readable medium 860 may include instructions to perform or facilitate some or all of the device functionality described herein. For instance, in the illustrated embodiment, program instructions 870 include a controller module 872, calculation and decision module 874 and an alert module 876.

The controller module 872 can include instructions for operating the target modification system 810, for example, the signal source 812 and collection magnet 814. For example, the controller 872 may activate signal source 812 and/or collection magnet 812 during each modification period in a set of pre-set modification periods. In particular, the controller module 872 can include instructions for controlling the signal source 812 and collection magnet 812 to transmit a modification signal at preset times in order to modify targets in a proximate portion of subsurface vasculature.

The controller module 872 can also include instructions for operating a user interface 820. For example, controller module 872 may include instructions for displaying data about the target modification system 810 and analyzed by the calculation and decision module 874, or for displaying one or more alerts generated by the alert module 876. Further, controller module 872 may include instructions to execute certain functions based on inputs accepted by the user interface 820, such as inputs accepted by one or more buttons disposed on the user interface.

Communication interface 830 may also be operated by instructions within the controller module 872, such as instructions for sending and/or receiving information via an antenna, which may be disposed on or in the wearable device 800. The communication interface 830 can optionally include one or more oscillators, mixers, frequency injectors, etc. to modulate and/or demodulate information on a carrier frequency to be transmitted and/or received by the antenna. In some examples, the wearable device 800 is configured to indicate an output from the processor by modulating an impedance of the antenna in a manner that is perceivable by a remote server or other remote computing device.

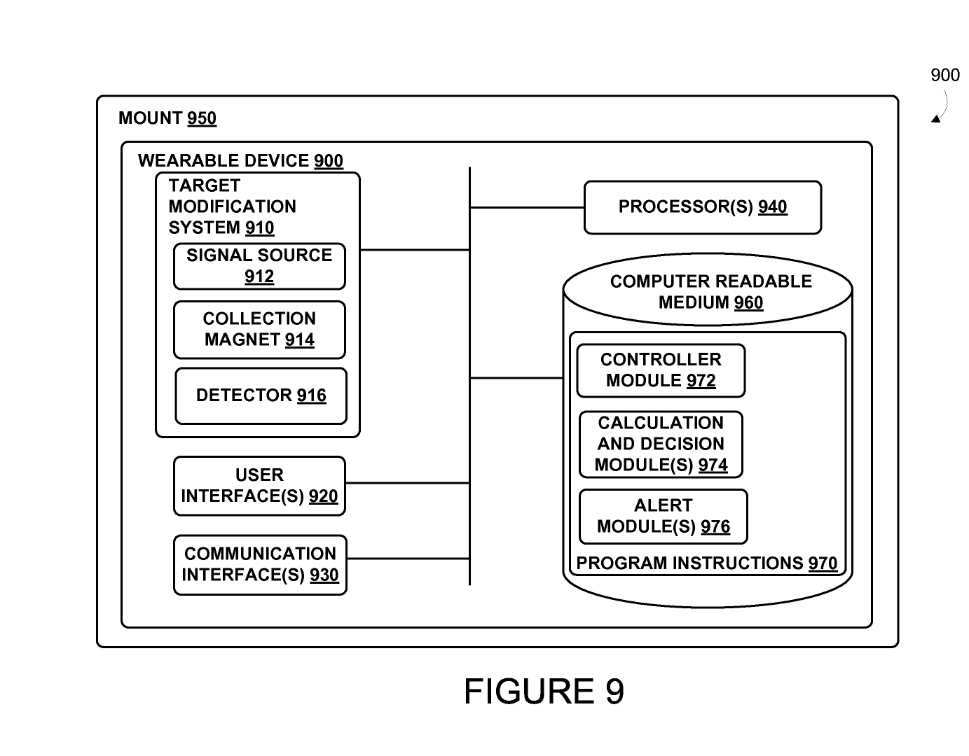

FIG. 9 is a simplified block diagram illustrating the components of a wearable device 900, according to an example embodiment. Wearable device 900 is the same as wearable device 800 in all respects, except that the target modification system 910 of wearable device 900 further includes a detector 916. Wearable device 900 includes a target modification system 910, which includes a signal source 912, a collection magnet 914 (if provided) and a detector 916, a user interface 920, a communication interface 930, a processor 940 and a computer readable medium 960 on which program instructions 970 are stored. All of the components of wearable device 900 may be provided on a mount 950. In this example, the program instructions 970 may include a controller module 972, a calculation and decision module 974 and an alert module 976 which, similar to the example set forth in FIG. 8, include instructions to perform or facilitate some or all of the device functionality described herein.

As described above, detector 916 may include any detector capable of detecting at least one physiological parameter, which could include any parameters that may relate to the health of the person wearing the wearable device. For example, the detector 916 could be configured to measure blood pressure, pulse rate, skin temperature, etc. At least one of the detectors 916 could be configured to non-invasively measure one or more targets in blood circulating in the subsurface vasculature proximate to the wearable device. In some examples, detector 916 may include one or more of an optical (e.g., CMOS, CCD, photodiode), acoustic (e.g., piezoelectric, piezoceramic), electrochemical (voltage, impedance), thermal, mechanical (e.g., pressure, strain), magnetic, or electromagnetic (e.g., RF, magnetic resonance) sensor.

In this example, the signal source 912 is configured to transmit an interrogating signal that can penetrate the wearer's skin into the portion of subsurface vasculature, for example, into a lumen of the subsurface vasculature. The interrogating signal can be any kind of signal, such as an electromagnetic, magnetic, optic, acoustic, thermal, or mechanical signal, that results in a response signal that can be used to measure a physiological parameter or, more particularly, that can detect the binding of the clinically-relevant target to the functionalized particles. In one example, the interrogating signal is an electromagnetic pulse (e.g., a radio frequency (RF) pulse) and the response signal is a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the particles by physical motion in a manner different from the background, making them easier to detect. In a further example, the interrogating signal is an electromagnetic radiation signal. In some examples, the functionalized particles include a fluorophore. The interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector. In some examples, this interrogation signal is the same as the modification signal used to change the target.

In some cases, the interrogation signal is not necessary to measure one or more of the physiological parameters. For example, the functionalized particles include an autofluorescent or luminescent marker, such as a fluorophore, that will automatically emit a response signal indicative of the binding of the clinically-relevant target to the functionalized particles, without the need for a modifying or interrogating signal or other external stimulus. In some examples, the functionalized particles may include a chemo-luminescent marker configured to produce a response signal in the form of fluorescence radiation produced in response to a chemical reaction initiated, at least in part, to the binding of the target analyte to the particle.

Calculation and decision module 972 may additionally include instructions for receiving data from the target modification system 910 in the form of a signal from the detector 916, analyzing the data to determine if the target analyte is present or absent, quantify the measured physiological parameter(s), such as concentration of a target analyte, and analyzing the data to determine if a medical condition is indicated. In particular, the calculation and decision module 972 may include instructions for determining, for each preset modification time, a target concentration of a clinically-relevant target analyte. The controller module 972 could then activate the signal source 912 and collection magnet 914 to effect a reduction in the concentration of the target. The controller module 962 could also continue to determine the concentration of the target based on the signal detected by the detector 916 during the activation of the signal source 912. The calculation and decision module 974 could then use this information to determine whether to continue or to discontinue the modification of the target analyte by the signal source 912. Further, the calculation and decision module 974 could quantify the measured physiological parameter(s), such as concentration of a target, and determine whether a medical condition is indicated based on at least the corresponding concentration of the clinically-relevant target. The preset modification and measurement times may be set to any period and, in one example, are about one hour apart.

The program instructions of the calculation and decision module 974 may, in some examples, be stored in a computer-readable medium and executed by a processor located external to the wearable device. For example, the wearable device could be configured to collect certain data regarding physiological parameters from the wearer and then transmit the data to a remote server, which may include a mobile device, a personal computer, the cloud, or any other remote system, for further processing.

The computer readable medium 960 may further contain other data or information, such as medical and health history of the wearer of the device that may be necessary in determining whether a medical condition is indicated. Further, the computer readable medium 960 may contain data corresponding to certain target analyte baselines, above or below which a medical condition is indicated. The baselines may be pre-stored on the computer readable medium 960, may be transmitted from a remote source, such as a remote server, or may be generated by the calculation and decision module 974 itself. The calculation and decision module 974 may include instructions for generating individual baselines for the wearer of the device based on data collected over a certain number of measurement periods. For example, the calculation and decision module 974 may generate a baseline concentration of a target blood analyte for each of a plurality of measurement periods by averaging the target concentration at each of the measurement periods measured over the course of a few days, and store those baseline concentrations in the computer readable medium 960 for later comparison. Baselines may also be generated by a remote server and transmitted to the wearable device 900 via communication interface 930. The calculation and decision module 974 may also, upon determining that a medical condition is indicated, generate one or more recommendations for the wearer of the device based, at least in part, on consultation of a clinical protocol. Such recommendations may alternatively be generated by the remote server and transmitted to the wearable device.

In some examples, the collected physiological parameter data, baseline profiles, history of target modification system activity, health state information input by device wearers and generated recommendations and clinical protocols may additionally be input to a cloud network and be made available for download by a wearer's physician. Trend and other analyses may also be performed on the collected data, such as physiological parameter data and health state information, in the cloud computing network and be made available for download by physicians or clinicians.

Further, physiological parameter and health state data from individuals or populations of device wearers may be used by physicians or clinicians in monitoring efficacy of a drug or other treatment. For example, high-density, real-time data may be collected from a population of device wearers who are participating in a clinical study to assess the safety and efficacy of a developmental drug or therapy. Such data may also be used on an individual level to assess a particular wearer's response to a drug or therapy. Based on this data, a physician or clinician may be able to tailor a drug treatment to suit an individual's needs. The therapy could include the modification or destruction of the target by the signal source in the wearable device.

In response to a determination by the calculation and decision module 974 that a medical condition is indicated, the alert module 976 may generate an alert via the user interface 920. The alert may include a visual component, such as textual or graphical information displayed on a display, an auditory component (e.g., an alarm sound), and/or tactile component (e.g., a vibration). The textual information may include one or more recommendations, such as a recommendation that the wearer of the device contact a medical professional, seek immediate medical attention, or administer a medication.

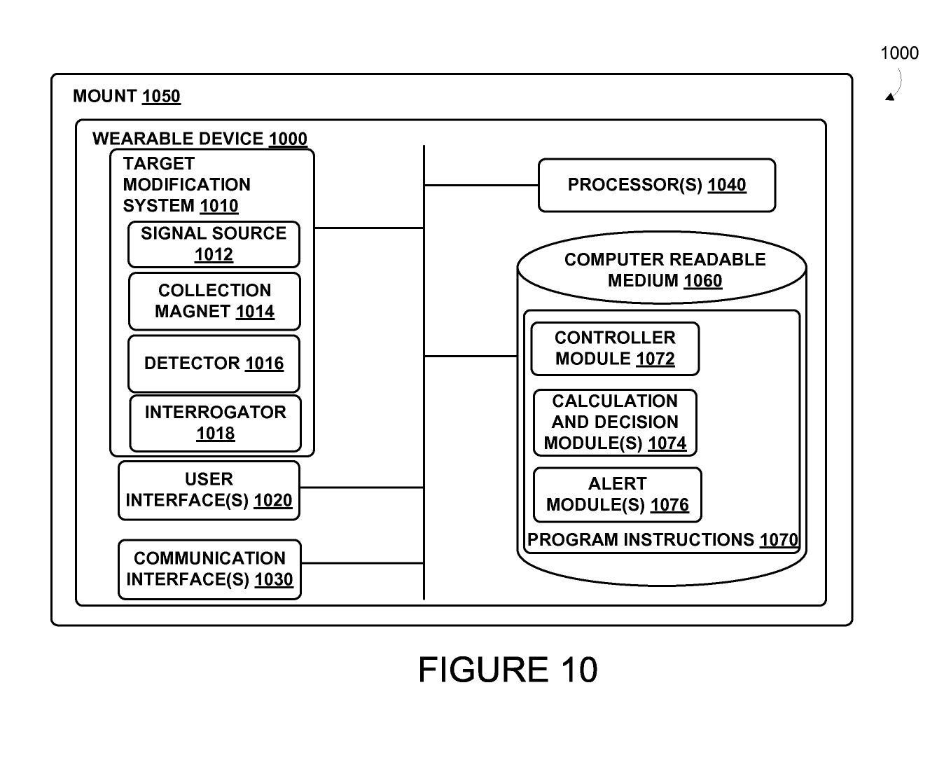

FIG. 10 is a simplified block diagram illustrating the components of a wearable device 1000, according to an example embodiment. Wearable device 1000 is the same as wearable device 900 in all respects, except that the target modification system 1010 of wearable device 1000 further includes an interrogator 1018. Wearable device 1000 includes a target modification system 1010, which includes a signal source 1012, a collection magnet 1014 (if provided), a detector 1016 and an interrogator 1018, a user interface 1020, a communication interface 1030, a processor 1040 and a computer readable medium 1060 on which program instructions 1070 are stored. All of the components of wearable device 1000 may be provided on a mount 1050. In this example, the program instructions 1070 may include a controller module 1072, a calculation and decision module 1074 and an alert module 1076 which, similar to the example set forth in FIG. 9, include instructions to perform or facilitate some or all of the device functionality described herein.

In this example, the interrogator 1018 is configured to transmit an interrogating signal that can penetrate into the portion of subsurface vasculature, for example, into a lumen of the subsurface vasculature. The interrogating signal can be any kind of signal that is benign to the wearer, such as electromagnetic, magnetic, optic, acoustic, thermal, mechanical, and results in a response signal that can be used to measure a physiological parameter or, more particularly, that can detect the binding of the clinically-relevant target to the functionalized particles. This interrogation is distinct from the transmissions of the signal source 1012; signal source 1012 transmits signals which are able to modify the target analyte. The signals emitted from the interrogator 1018 enable the detection of the target analyte by causing the detector 1016 to receive a signal related to the target analyte. In one example, the interrogating signal is an electromagnetic pulse (e.g., a radio frequency (RF) pulse) and the response signal is a magnetic resonance signal, such as nuclear magnetic resonance (NMR). In another example, the interrogating signal is a time-varying magnetic field, and the response signal is an externally-detectable physical motion due to the time-varying magnetic field. The time-varying magnetic field modulates the particles by physical motion in a manner different from the background, making them easier to detect. In a further example, the interrogating signal is an electromagnetic radiation signal. In some examples, the functionalized particles include a fluorophore. The interrogating signal may therefore be an electromagnetic radiation signal with a wavelength that can excite the fluorophore and penetrate the skin or other tissue and subsurface vasculature (e.g., a wavelength in the range of about 500 to about 1000 nanometers), and the response signal is fluorescence radiation from the fluorophore that can penetrate the subsurface vasculature and tissue to reach the detector.

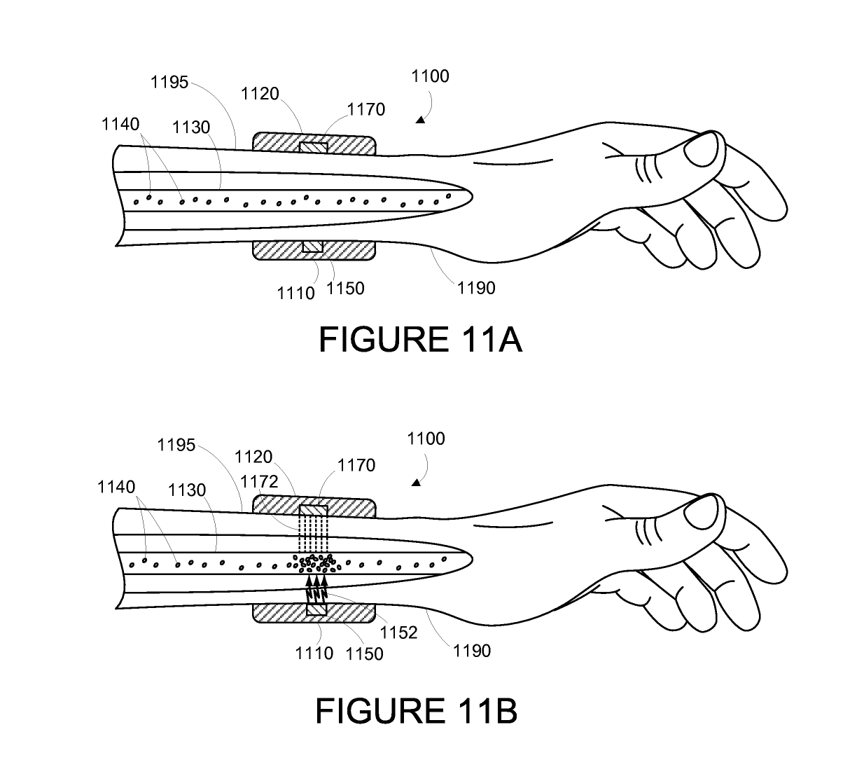

FIGS. 11A-11B, 12A-12B, 13A-13B, and 14A-14B are partial cross-sectional side views of a human wrist illustrating the operation of various examples of a wrist-mounted device. In the example shown in FIGS. 11A and 11B, the wrist-mounted device 1100 includes a modification platform 1110 mounted on a strap or wrist-band 1120 and oriented on the anterior side 1190 of the wearer's wrist and the collection magnet 1170 is disposed on the anterior side 1195 of the wearer's wrist. Modification platform 1110 is positioned over a portion of the wrist where subsurface vasculature 1130 is easily observable. Functionalized particles 1140 have been introduced into a lumen of the subsurface vasculature by one of the means discussed above. In this example, modification platform 1110 includes a signal source 1150. FIG. 11A illustrates the state of the subsurface vasculature when the wrist-mounted device 1100 is inactive. The state of the subsurface vasculature during a modification period is illustrated in FIG. 11B. At this time, collection magnet 1170 generates a magnetic field 1172 sufficient to cause functionalized magnetic particles 1140 present in a lumen of the subsurface vasculature 1130 to collect in a region proximal to the magnet 1170. At this time, signal source 1150 is transmitting a modification signal 1152 into the portion of subsurface vasculature. The modification signal 1152 causes a chemical or physical change in a clinically relevant target analyte bound to the functionalized particles 1140 present in the subsurface vasculature 1130.

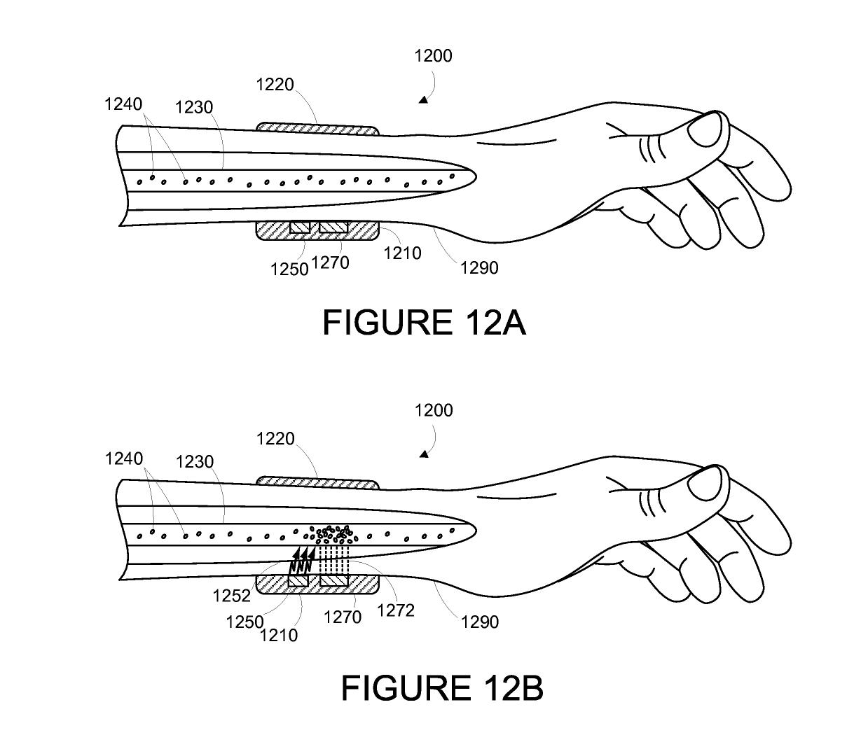

Similar to the system depicted in FIGS. 11A and 11B, FIGS. 12A and 12B illustrate a wrist-mounted device 1200 including a modification platform 1210 mounted on a strap or wristband 1220 and oriented on the anterior side 1290 of the wearer's wrist. In this example, modification platform 1210 includes a signal source 1250 and a collection magnet 1270. FIG. 12A illustrates the state of the subsurface vasculature 1230 when measurement device 1200 is inactive. The state of the subsurface vasculature when modification device 1200 is active during a modification period is illustrated in FIG. 12B. At this time, collection magnet 1270 generates a magnetic field 1272 sufficient to cause functionalized magnetic particles 1240 present in a lumen of the subsurface vasculature 1230 to collection in a region proximal to the magnet 1270. Signal source 1250 transmits a modification signal 1252 into the portion of subsurface vasculature 1230. The modification signal 1252 causes a chemical or physical change in a clinically relevant target bound to the functionalized particles 1240 present in the subsurface vasculature 1230.

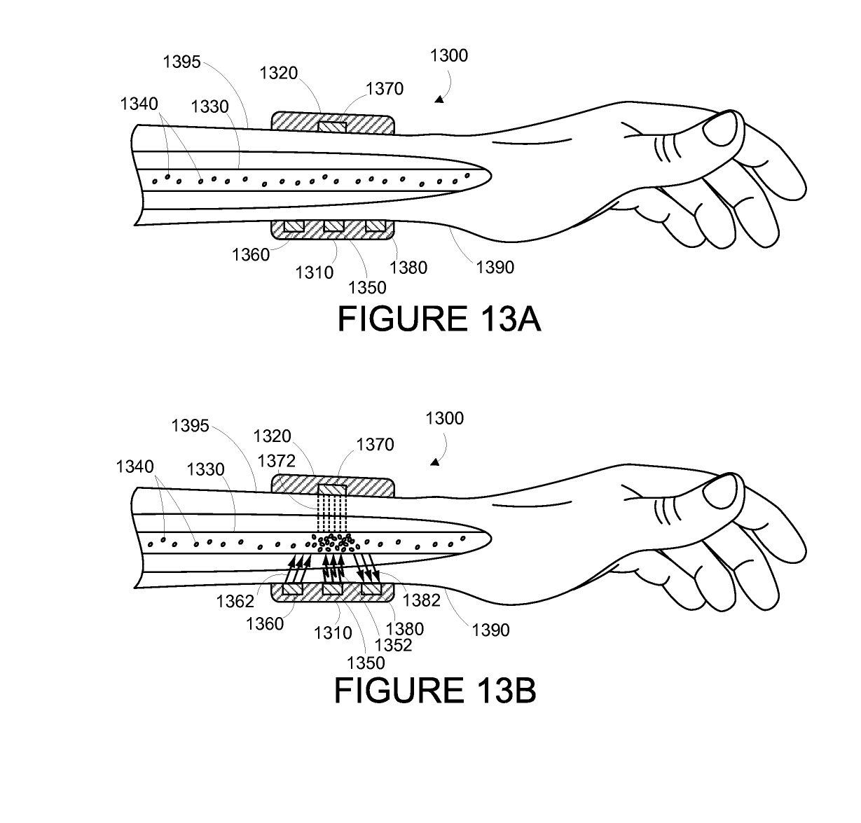

FIGS. 13A and 13B illustrate a further embodiment of a wrist-mounted device 1300 having a measurement platform 1310 disposed on a strap 1320, wherein the signal source 1350, an interrogation signal source 1360, and a detector 1380 are positioned on the posterior side 1390 of the wearer's wrist and the collection magnet 1370 is disposed on the anterior side 1395 of the wearer's wrist. Similar to the embodiments discussed above, FIG. 13A illustrates the state of the subsurface vasculature 1330 when modification device 1300 is inactive. The state of the subsurface vasculature 1330 when modification device 1300 is active during a modification and measurement period is illustrated in FIG. 13B. At this time, collection magnet 1370 generates a magnetic field 1372 sufficient to cause functionalized magnetic particles 1340 present in a lumen of the subsurface vasculature 1330 to collect in a region proximal to the magnet 1370. Interrogation signal source 1360 transmits an interrogating signal 1362 into the portion of subsurface vasculature and detector 1380 is receiving a response signal 1382 generated in response to the interrogating signal 1362. The response signal 1382 is related to the binding of a clinically relevant target present in the subsurface vasculature to the functionalized magnetic particles 1340. Signal source 1350 transmits a modification signal 1352 into the portion of subsurface vasculature 1330. The modification signal 1352 causes a chemical or physical change in a clinically relevant target bound to the functionalized particles 1340 present in the subsurface vasculature 1330. The generation of the modification and interrogation signals during respective modification and interrogation periods may be at the same time or at different times.

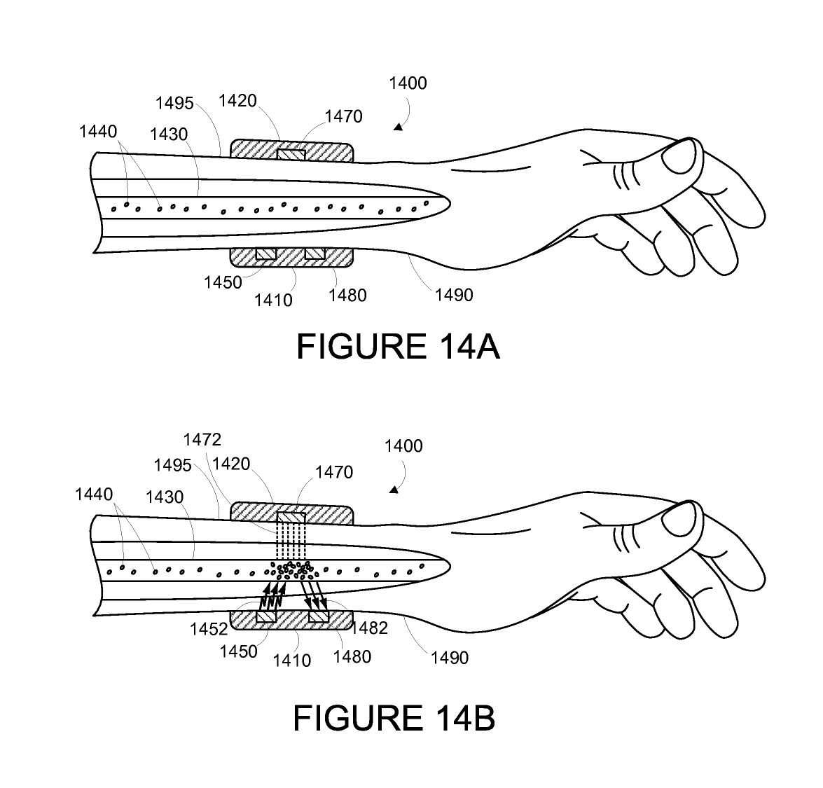

FIGS. 14A and 14B illustrate a further embodiment of a wrist-mounted device 1400 having a modification platform 1410 disposed on a strap 1420, wherein the signal source 1450 and a detector 1480 are positioned on the posterior side 1490 of the wearer's wrist and the collection magnet 1470 is disposed on the anterior side 1495 of the wearer's wrist. Similar to the embodiments discussed above, FIG. 14A illustrates the state of the subsurface vasculature 1430 when modification device 1400 is inactive. The state of the subsurface vasculature 1430 when measurement device 1400 is active during a modification and measurement period is illustrated in FIG. 14B. At this time, collection magnet 1470 generates a magnetic field 1472 sufficient to cause functionalized magnetic particles 1440 present in a lumen of the subsurface vasculature 1430 to collect in a region proximal to the magnet 1470. Signal source 1450 transmits a modification signal 1452 into the portion of subsurface vasculature 1430. The modification signal 1452 causes a chemical or physical change in a clinically relevant target bound to the functionalized particles 1440 present in the subsurface vasculature 1430. Detector 1480 is receiving a response signal 1482 generated in response to the modification signal 1452. The response signal 1482 is related to the binding of a clinically relevant target present in the subsurface vasculature 1430 to the functionalized magnetic particles 1440. As described above, in some embodiments, a transmitted signal 1452 may not be necessary to generate a response signal 1482 related to the binding of a target to the functionalized magnetic particles 1440. Additionally, the signal source 1450 may be configured to transmit different types of signals such that some signals modify the target and other signals enable detection of the target. Further, these different signals may occur at different timing according to use of the device; for example, the detection signals may be generated at a regular interval (e.g., 1 hour) over the course of an entire day, while the modification signal is only generated during one or two therapeutic periods during a day.

FIGS. 11B, 12B, 13B and 14B illustrate example configurations of wrist-mounted devices (1100, 1200, 1300, 1400). They show configurations of modification signal sources (1150, 1250, 1350, 1450), collection magnets (1170, 1270, 1370, 1470), interrogation signal sources (1360), detectors (1380, 1480), magnetic fields (1172, 1272, 1372, 1472), the paths of modification signals (1152, 1252, 1352, 1452), interrogation signals (1362), and response signals (1382, 1482) relative to each other and to the subsurface vasculature (1130, 1230, 1330, 1430) which are meant as illustration only. The components listed above may be configured as described above relative to the posterior (1190, 1290, 1390, 1490) and anterior (1195, 1295, 1395, 1495) sides of the wearer's wrist or in other configurations according to an application, as will be evident to one skilled in the art. Further, the various components may be configured to direct signals into or detects signals from the same area of the subsurface vasculature (1130, 1230, 1330, 1430) as described in FIGS. 11, 12, 13, and 14 or different areas of the subsurface vasculature according to a specific application.

### IV. ILLUSTRATIVE FUNCTIONALIZED PARTICLES

In some examples, the wearable devices described above cause the modification of the targets by affecting functionalized particles, for example, microparticles or nanoparticles, which have become bound to a clinically-relevant target. The particles can be functionalized by covalently attaching a bioreceptor designed to selectively bind or otherwise recognize a particular clinically-relevant target. For example, particles may be functionalized with a variety of bioreceptors, including antibodies, nucleic acids (DNA, siRNA), low molecular weight ligands (folic acid, thiamine, dimercaptosuccinic acid), peptides (RGD, LHRD, antigenic peptides, internalization peptides), proteins (BSA, transferrin, antibodies, lectins, cytokines, fibrinogen, thrombin), polysaccharides (hyaluronic acid, chitosan, dextran, oligosaccharides, heparin), polyunsaturated fatty acids (palmitic acid, phospholipids), or plasmids. The functionalized particles can be introduced into the person's blood stream by injection, ingestion, inhalation, transdermal application, or in some other manner.

The clinically-relevant target could be any substance that, when present in the blood, or present at a particular concentration or range of concentrations, may directly or indirectly cause an adverse medical condition. For example, the clinically-relevant target could be an enzyme, hormone, protein, other molecule, or even whole or partial cells. In one relevant example, certain proteins have been implicated as a partial cause of Parkinson's disease; the development of Parkinson's disease may be prevented or retarded by providing particles functionalized with a bioreceptor that will selectively bind to this target. These bound particles may then be used, in combination with a wearable device as described above, to modify or destroy the target protein. As a further example, the target could be cancer cells; by selectively targeting and then modifying or destroying the cancer cells, the spread of cancer may be diminished.

Modification or destruction as described herein refers to causing a change in the target such that the target's ability to cause the adverse medical condition is reduced. The change may consist of denaturing a protein, lysing a cell, a chemical change, or a variety of other effects familiar to one skilled in the art. The change in the target may be caused by localized heating of the target due to energy transmitted by a wearable device and transduced into localized heating by a functionalized particle bound to the target. For example, the functionalized particle could be magnetic and have a resonance frequency; transmitting an RF pulse or time-varying magnetic field at the resonance frequency could cause localized heating near the functionalized particle which could cause a change in a target bound to the functionalized particle. The changed target may still be able to cause the adverse medical condition, but to a lesser degree than before the change. The changed target may also cause adverse medical conditions or other side effects different from the original adverse medical condition.

Further, the particles may be formed from a paramagnetic or ferromagnetic material or be functionalized with a magnetic moiety. An external magnet may be used to locally collect the particles in an area of subsurface vasculature during a modification period. Such collection may reduce the energy of the modification signal necessary to modify or destroy an amount of the target by concentrating the target near the modification signal source. In another example, the magnetic properties of the particles can be exploited in magnetic resonance detection schemes to enable detection of the concentration of the target.

The particles may be made of biodegradable or non-biodegradable materials. For example, the particles may be made of polystyrene. Non-biodegradable particles may be provided with a removal means to prevent harmful buildup in the body. Generally, the particles may be designed to have a long half-life so that they remain in the vasculature or body fluids over several modification periods. Depending on the lifetime of the particles, however, the user of the wearable device may periodically introduce new batches of functionalized particles into the vasculature or body fluids.

Further, the particles may be designed to either reversibly or irreversibly bind to the target. For example, if the modification or destruction of the target, as described above, also involves the destruction or disabling of the particles, they may irreversibly bind to the target. In other examples, the particles may be designed to release the target after it has been modified or destroyed, either automatically or in response to an external or internal stimulus.

More than one type of functionalized particle may be used. For example, one particle may bind to a target, causing the target to expose a binding site which was not normally exposed. A second type of particle may then bind to the exposed binding site. This second particle may then allow the target to be modified or destroyed by a wearable device. Further, one or both of the particles may be magnetic, as described above, allowing the target to be concentrated in the lumen of a subsurface vasculature proximate to a wearable device. One of the types of particle could be also be configured to bind to another type of particle rather than to a target. The use of multiple types of particles could also be used to increase the specificity of the modification or destruction of the target, avoiding damage to non-harmful analytes that might be damaged when using a less specific single type of particle.

If the modification or destruction of the target also causes the destruction of one of the bound particles, the use of more than one particle may be used to minimize cost. For example, the target-specific particle may be expensive, so a second particle is used which is less expensive and which binds to the target once the first particle has bound to the target. This second particle allows the wearable device to modify or destroy the target. This modification or destruction results in the disabling of the second particle, but the first particle is able to unbind from the changed target and bind to another instance of the unchanged target. The supply of the second functionalized particle in the blood can be replenished more frequently and at a lower cost than the first functionalized particle.

Those of skill in the art will understand the term “particle” in its broadest sense and that it may take the form of any fabricated material, a molecule, tryptophan, a virus, a phage, etc. Further, a particle may be of any shape, for example, spheres, rods, non-symmetrical shapes, etc. The particles can have a diameter that is less than about 20 micrometers. In some embodiments, the particles have a diameter on the order of about 10 nm to 1 μm. In further embodiments, small particles on the order of 10-100 nm in diameter may be assembled to form a larger “clusters” or “assemblies” on the order of 1-10 micrometers. In this arrangement, the assemblies would provide the effects of a larger particle, but would be deformable, thereby preventing blockages in smaller vessels and capillaries.

Further, the terms “binding”, “bound”, and related terms used herein are to be understood in their broadest sense to include any interaction between the receptor and the target or another functionalized particle such that the interaction allows the target to be modified or destroyed by energy emitted from a wearable device.

In some examples, the wearable devices described above obtain some health-related information by detecting the binding of a clinically-relevant target. Binding of the functionalized particles to a target may be detected with or without a stimulating signal input. For example, some particles may be functionalized with compounds or molecules, such as fluorophores or autofluorescent, luminescent or chemo-luminescent markers, which generate a responsive signal when the particles bind to the target without the input of a stimulus. In other examples, the functionalized particles may produce a different responsive signal in their bound versus unbound state in response to an external stimulus, such as an electromagnetic, acoustic, optical, or mechanical energy. Further, this external stimulus may be related to the stimulus which modifies or destroys the target or may be an unrelated stimulus.

### V. ILLUSTRATIVE METHODS FOR CHANGING A TARGET ANALYTE IN THE BLOOD TO REDUCE AN ADVERSE HEALTH EFFECT

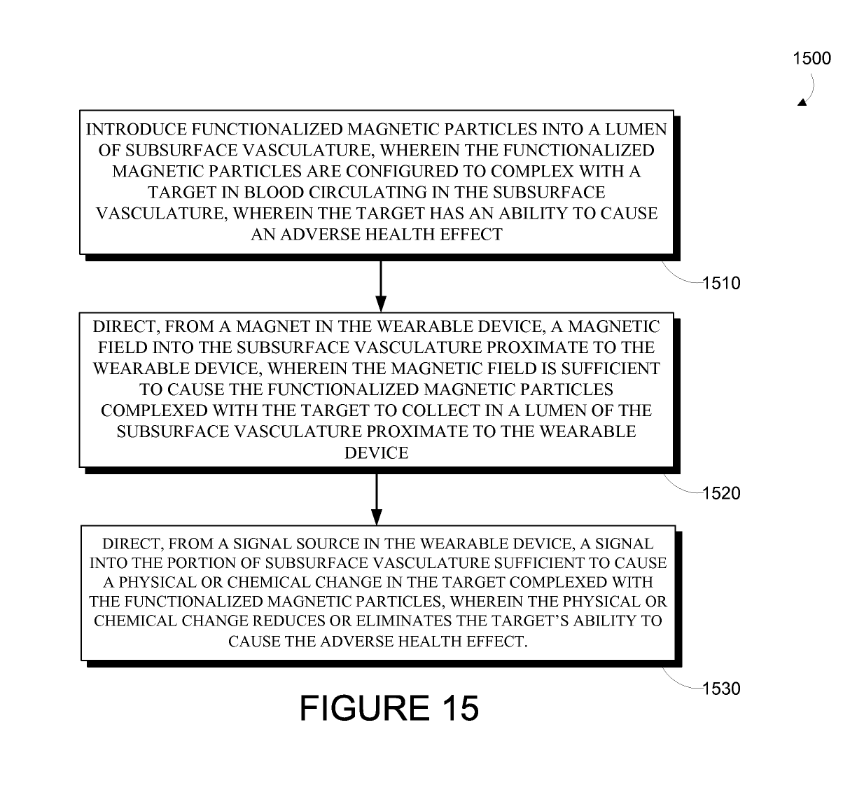

FIG. 15 is a flowchart of an example method 1500 for operating a wearable device to non-invasively reduce the amount of a harmful target in the blood. Functionalized magnetic particles are introduced into a lumen of a subsurface vasculature, such that the functionalized magnetic particles are configured to complex with a clinically-relevant target in blood circulating in the subsurface vasculature, where the target is able to cause an adverse health effect (1510). A magnetic field is directed by a wearable device into the subsurface vasculature proximate to the wearable device such that the functionalized magnetic particles complexed with the target collect in the lumen of the subsurface vasculature proximate to the wearable device (1520). A signal source in the wearable device then directs a signal into the subsurface vasculature proximate to the device sufficient to cause a physical or chemical change in the target complexed with the functionalized magnetic particles, such that the target's ability to cause the adverse health effect is reduced (1530).

The introduction of functionalized magnetic particles into a lumen of a subsurface vasculature, such that the particles are configured to complex with a clinically-relevant target in blood circulating in the subsurface vasculature, where the target is able to cause an adverse health effect (1510) could consist of injecting a solution containing functionalized magnetic particles into the bloodstream of a user. It could also consist of intramuscular, intraperitoneal, or subcutaneous injection, ingestion, inhalation, a transdermal patch or spray, or any other method familiar to one skilled in the art. The configuration of the particles to complex with the target includes any method by which the particles may be made to specifically bind to the target. The term “bind” is understood in its broadest sense to also include any interaction between the clinically relevant target and the functionalized magnetic particles sufficient to allow the target to be modified or destroyed by a signal directed from a wearable device. For example, it could include covalently attaching a receptor that specifically binds or otherwise recognizes a particular clinically-relevant target. The functionalized receptor can be an antibody, peptide, nucleic acid, phage, bacteria, virus, or any other molecule with a defined affinity for a target. Alternatively, the particle itself may be a virus or a phage with an inherent affinity for certain analytes that has been functionalized to include a magnetic particle or moiety. The target could be a protein, a hormone, a cell, a plaque, or any other substance in the blood which could cause an adverse health reaction.

Directing a magnetic field into the subsurface vasculature proximate to the wearable device such that the functionalized magnetic particles complexed to the target collect in the lumen of the subsurface vasculature proximate to the wearable device (1520) could include energizing an electromagnet built into the wearable device. It could also include incorporating a permanent magnet into the wearable device or any other method of creating a magnetic field familiar to one skilled in the art. The field could be constant or variable in time.

A signal source in the wearable device directing a signal into the subsurface vasculature proximate to the device sufficient to cause a physical or chemical change in the target complexed with the functionalized magnetic particles, such that the target's ability to cause the adverse health effect is reduced (1530) could include directing a time-varying magnetic field into the lumen of the subsurface vasculature of sufficient energy to excite the functionalized magnetic particles such that the target is damaged. Transmitting from the signal source a time-varying magnetic field at a resonance frequency of the particles could result in localized heating of the target, causing the modification or destruction of the target. The signal could also include an acoustic pulse, a radio-frequency pulse, an electromagnetic pulse, a time-varying magnetic field, an infrared light pulse, or any other directed energy familiar to one skilled in the art which is capable of selectively modifying or destroying targets complexed with functionalized magnetic particles. In another example, the signal source transmits a light pulse, which causes a localized heating of the target due to a photoacoustic effect. Modification or destruction as described includes any changes in the target such that the adverse health effect is reduced. However, the changed target (or other products of the change) may exhibit other adverse health effects or side effects.

Further, the wearable device may be configured to measure one or more physiological parameters of the wearer that may relate to the health of the person wearing the wearable device. For example, the wearable device could include sensors for measuring blood pressure, pulse rate, skin temperature, or any other parameters familiar to one skilled in the art. At least some of the physiological parameters may be obtained by the wearable device non-invasively detecting and/or measuring one or more target analytes in blood circulating in subsurface vasculature proximate to the wearable device. This could be accomplished by detecting a signal emitted from a functionalized magnetic particle complexed with the target; this signal could be emitted in response to the modifying or destroying signal described above or may be in response to another signal transmitted into the subsurface vasculature with the purpose of detecting the presence or concentration of the target. The signal may also be emitted spontaneously, without any incoming interrogating signal (e.g., due to an autoflourescent or chemiluminescent functionalization or some other method familiar to one skilled in the art).

The functionalized magnetic particles may be configured to allow for the binding of other functionalized particles to the first functionalized magnetic particle or to the target. These other particles may allow for detection of the target by a wearable device, increase the specificity of the binding with the target, or any other useful function related to the target and its adverse health effects. Other compounds or molecules, such as fluorophores or autofluorescent or luminescent markers, which may assist in interrogating the particles in vivo, may also be attached to one or more of the types of particles.

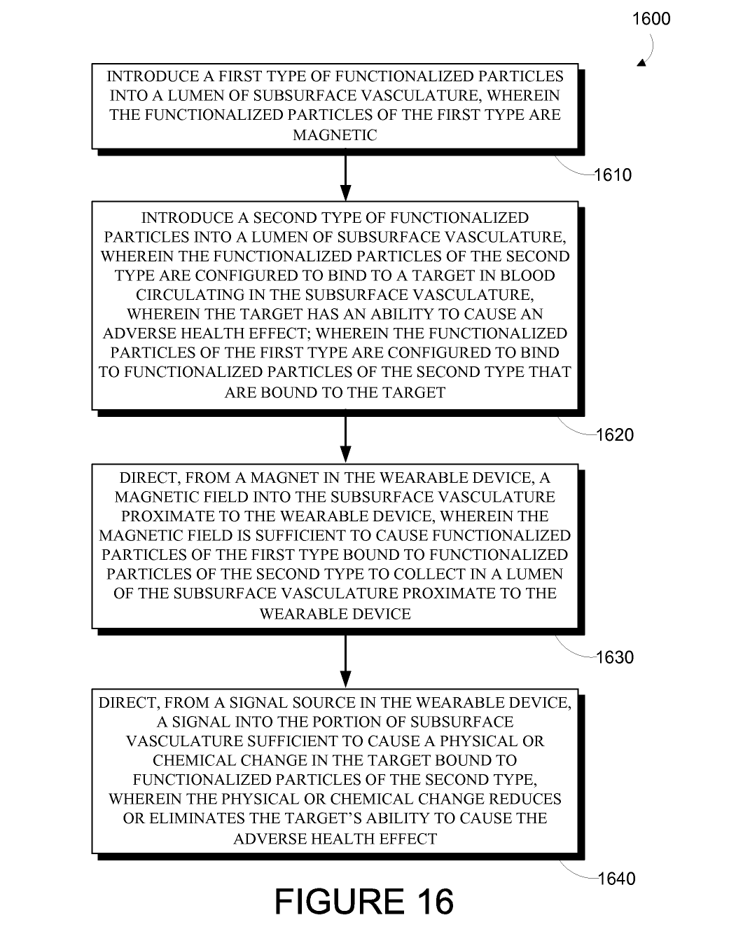

FIG. 16 is a flowchart of an example method 1600 for operating a wearable device to non-invasively reduce the amount of a harmful target in the blood. Functionalized magnetic particles are introduced into a lumen of a subsurface vasculature (1610). A second type of functionalized particles is then introduced into a lumen of the subsurface vasculature, such that the second type of functionalized particles is configured to bind to a target in the blood that is able to cause an adverse health effect, such that functionalized particles of the first type are configured to bind to functionalized particles of the second type which are bound to the target (1620). A magnetic field is directed into the subsurface vasculature proximate to the wearable device such that the functionalized magnetic particles bound to the functionalized particles of the second type collect in the lumen of the subsurface vasculature proximate to the wearable device (1630). A signal source in the wearable device then directs a signal into the subsurface vasculature proximate to the device sufficient to cause a physical or chemical change in the target bound to the functionalized particles of the second type, such that the target's ability to cause the adverse health effect is reduced (1640).

The introduction of the first type and second type of functionalized magnetic particles into a lumen of a subsurface vasculature (1610) could involve any of the techniques as described above. The functionalized magnetic particle may include a ferromagnetic, paramagnetic or some other magnetic material functionalized to bind to other particles or targets. The functionalized magnetic particle may alternatively be some other particle which has been functionalized with a magnetic material or magnetic moiety.

The configuration of the second particles that bind to the target includes any method by which the particles may be made to specifically bind to the target. The term “bind” is understood in its broadest sense to also include any interaction between the clinically relevant target and the second functionalized particles sufficient to allow the target to be modified or destroyed by a signal directed from a wearable device when the second functionalized particles or the target are bound to the first functionalized magnetic particle. For example, it could include covalently attaching a receptor that specifically binds or otherwise recognizes a particular clinically-relevant. The functionalized receptor can be an antibody, peptide, nucleic acid, phage, bacteria, virus, or any other molecule with a defined affinity for a target. Alternatively, the particle itself may be a virus or a phage with an inherent affinity for certain analytes which has been functionalized to allow the binding of the first functionalized magnetic particle. The target could be a protein, a hormone, a cell, a plaque, or any other substance in the blood which could cause an adverse health reaction.

The first functionalized magnetic particle may bind to the second functionalized particle or directly to the target. The binding of the second particle to the target may cause a change in the shape of the target such that the first particle can selectively bind to the target. Alternatively, binding to the target may cause a change in the shape of the second particle such that the first particle can selectively bind to the second particle. Other configurations of the first and second particles may be used which cause other methods of binding between the target and the first and second particles such that the target is collected in a lumen of the subsurface vasculature in the proximity of a wearable device, such that an energy directed from the wearable device is able to change the target and reduce the harmful health effects of the target.

Directing a magnetic field into the subsurface vasculature proximate to the wearable device such that the first functionalized magnetic particles complexed to the second functionalized particles collect in the lumen of the subsurface vasculature proximate to the wearable device (1630) could include energizing an electromagnet built into the wearable device. It could also include incorporating a permanent magnet into the wearable device or any other method of creating a magnetic field familiar to one skilled in the art. The field could be constant or variable in time.

A signal source in the wearable device directing a signal into the subsurface vasculature proximate to the device sufficient to cause a physical or chemical change in the target bound to the second functionalized particles, such that the target's ability to cause the adverse health effect is reduced (1640) could include directing a time-varying magnetic field into the lumen of the subsurface vasculature of sufficient energy to excite the functionalized magnetic particles such that the target is damaged. Transmitting from the signal source a time-varying magnetic field at a resonance frequency of the particles could result in localized heating of the target, causing the modification or destruction of the target. The signal could also include an acoustic pulse, a radio-frequency pulse, an electromagnetic pulse, a time-varying magnetic field, an infrared light pulse, or any other directed energy familiar to one skilled in the art which is capable of selectively modifying or destroying targets complexed with functionalized magnetic particles. In another example, the signal source transmits a light pulse, which causes a localized heating of the target due to a photoacoustic effect. Modification or destruction as described includes any changes in the target such that the adverse health effect is reduced. However, the changed target (or other products of the change) may exhibit other adverse health effects or side effects.

Further, the wearable device may be configured to measure one or more physiological parameters of the wearer that may relate to the health of the person wearing the wearable device. For example, the wearable device could include sensors for measuring blood pressure, pulse rate, skin temperature, or any other parameters familiar to one skilled in the art. At least some of the physiological parameters may be obtained by the wearable device non-invasively detecting and/or measuring one or more target analytes in blood circulating in subsurface vasculature proximate to the wearable device.

### VI. CONCLUSION

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

Where example embodiments involve information related to a person or a device of a person, some embodiments may include privacy controls. Such privacy controls may include, at least, anonymization of device identifiers, transparency and user controls, including functionality that would enable users to modify or delete information relating to the user's use of a product.

Further, in situations in where embodiments discussed herein collect personal information about users, or may make use of personal information, the users may be provided with an opportunity to control whether programs or features collect user information (e.g., information about a user's medical history, social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

What is claimed is:

1. A wearable device, comprising:
a mount configured to mount the wearable device to an external body surface proximate to a portion of subsurface vasculature;
a magnet configured to direct a magnetic field into the portion of subsurface vasculature, wherein the magnetic field is sufficient to cause functionalized magnetic particles to collect in a lumen of the portion of the subsurface vasculature, wherein the functionalized magnetic particles are configured to complex with a target, and wherein the target has an ability to cause an adverse health effect;
a signal source configured to transmit a signal into the portion of subsurface vasculature sufficient to cause a physical or chemical change in the target complexed with the functionalized magnetic particles, wherein the physical or chemical change reduces or eliminates the target's ability to cause the adverse health effect.

2. The wearable device of claim 1, wherein the functionalized magnetic particles are configured to complex with the target by binding to the target.

3. The wearable device of claim 1, wherein the functionalized magnetic particles are configured to complex with the target by binding to other functionalized particles that are bound to the target.

4. The wearable device of claim 1, wherein the signal comprises an electromagnetic pulse, an acoustic pulse, or a time-varying magnetic field.

5. The wearable device of claim 1, wherein the signal comprises an infrared pulse.

6. The wearable device of claim 1, wherein the signal comprises a radio frequency pulse.

7. The wearable device of claim 1, further comprising:
a detector configured to detect a response signal transmitted from the portion of subsurface vasculature, wherein the response signal is related to complexing of the target to the functionalized magnetic particles.

8. The wearable device of claim 7, further comprising:
a signal source configured to transmit an interrogating signal into the portion of subsurface vasculature, wherein the response signal is generated in response to the interrogating signal.

9. A method, comprising:
introducing functionalized magnetic particles into a lumen of subsurface vasculature, wherein the functionalized magnetic particles are configured to complex with a target in blood circulating in the subsurface vasculature, wherein the target has an ability to cause an adverse health effect;
directing, from a magnet in the wearable device, a magnetic field into the subsurface vasculature proximate to the wearable device, wherein the magnetic field is sufficient to cause the functionalized magnetic particles complexed with the target to collect in a lumen of the subsurface vasculature proximate to the wearable device;
directing, from a signal source in the wearable device, a signal into the portion of subsurface vasculature sufficient to cause a physical or chemical change in the target complexed with the functionalized magnetic particles, wherein the physical or chemical change reduces or eliminates the target's ability to cause the adverse health effect.

10. The method of claim 9, wherein the signal comprises an electromagnetic pulse, an acoustic pulse, or a time-varying magnetic field.

11. The method of claim 10, wherein the signal comprises a radio frequency pulse.

12. The method of claim 10, wherein the signal comprises an infrared light pulse.

13. The method of claim 9, wherein the target comprises a protein, a hormone, or a cell.

14. The method of claim 9, wherein the functionalized magnetic particles are configured to complex with the target by binding to the target.

15. The method of claim 9, wherein the functionalized magnetic particles are configured to complex with the target by binding to other functionalized particles that are bound to the target.

16. The method of claim 9, further comprising automatically measuring, by the wearable device, one or more physiological parameters during each of a plurality of measurement periods, wherein at least one of the physiological parameters is measured by non-invasively detecting one or more analytes in blood circulating in subsurface vasculature proximate to the wearable device.

17. A method, comprising:
introducing a first type of functionalized particles into a lumen of subsurface vasculature, wherein the functionalized particles of the first type are magnetic;
introducing a second type of functionalized particles into a lumen of subsurface vasculature, wherein the functionalized particles of the second type are configured to bind to a target in blood circulating in the subsurface vasculature, wherein the target has an ability to cause an adverse health effect;
wherein the functionalized particles of the first type are configured to bind to functionalized particles of the second type that are bound to the target;
directing, from a magnet in the wearable device, a magnetic field into the subsurface vasculature proximate to the wearable device, wherein the magnetic field is sufficient to cause functionalized particles of the first type bound to functionalized particles of the second type to collect in a lumen of the subsurface vasculature proximate to the wearable device;
directing, from a signal source in the wearable device, a signal into the portion of subsurface vasculature sufficient to cause a physical or chemical change in the target bound to functionalized particles of the second type, wherein the physical or chemical change reduces or eliminates the target's ability to cause the adverse health effect.

18. The method of claim 17, wherein the functionalized particles of the second set undergo a conformational change exposing a binding site when bound to the target and wherein the functionalized particles of the first set are configured to bind to the binding site.

19. The method of claim 17, wherein the signal comprises an electromagnetic pulse.

20. The method of claim 19, wherein the electromagnetic pulse is a radio frequency pulse.
